(19) United States
(12) Patent Application Publication
(10) Pub. No.: US 2026/0096929 A1
(43) Pub. Date: Apr. 9, 2026

(54) Goggles with lens change systems and methods of using the same

(71) Applicant: POC Sweden AB, STOCKHOLM (SE)

(72) Inventors: Robin GRIEVES, STOCKHOLM (SE); Claes NELLESTAM, STOCKHOLM (SE); Magnus GUSTAVSSON, STOCKHOLM (SE); Oscar HUSS, STOCKHOLM (SE); Ilango LOURDUDOSS, STOCKHOLM (SE); Sofie BERGLUND, STOCKHOLM (SE); Tilda HÅLL, STOCKHOLM (SE)

(21) Appl. No.: 19/417,380

(22) Filed: Dec. 12, 2025

Related U.S. Application Data
(63) Continuation of application No. PCT/EP2023/067926, filed on Jun. 29, 2023.

(51) Int. Cl. A61F 9/02

(52) U.S. Cl. CPC A61F 9/025

(57) ABSTRACT

A pair of goggles includes a goggle frame, a lens assembly coupled to the goggle frame, and first and second retention features securing the lens assembly to the goggle frame at first and second locations to prevent decoupling of the lens assembly from the goggle frame. The lens assembly is removable from the goggle frame by actuating one of the retention features. A method of removing a lens assembly from a goggle frame includes opening a locking element to unlock first and second latch components of a closure assembly, unlatching the first and second latch components, pivoting the lens assembly away from the goggle frame about a hinge securing the lens assembly to the goggle frame; and after pivoting the lens assembly, laterally translating the lens assembly away from the hinge.

### TECHNICAL FIELD

The present disclosure generally relates to the field of closure assemblies. In particular, the present disclosure is directed to closure assemblies for goggles with lens change systems and methods of using the same.

### BACKGROUND

Wearing eyewear with the appropriate lenses for a given ambient condition is important for user comfort as well as safety. Various designs for eyewear with interchangeable lenses have been developed that employ combinations of mechanical and magnetic coupling arrangements to provide the ability to change the lenses.

### SUMMARY

In one implementation, the present disclosure is directed to a closure assembly. The closure assembly includes a first latch component that includes a socket; a second latch component that includes a first portion; a locking element coupled to the second latch component, a bottom surface of the locking element having at least one protrusion; wherein, when in a latched configuration, the first portion is inserted into and mechanically engages the socket; wherein, when in a locked configuration, the at least one protrusion of the locking element is disposed in a locking element recess extending between the first and second latch components to thereby lock the first and second latch components in the latched configuration.

In another implementation, the present disclosure is directed to a closure assembly. The closure assembly includes a first latch component that includes a cross member having inner and outer surfaces; a second latch component that includes a first surface and a laterally extending portion; a locking element coupled to the second latch component by a pivot connection, a bottom surface of the locking element having at least one protrusion; wherein, when in a latched configuration, the first surface engages the inner surface of the cross member, and the outer surface of the cross member and the laterally extending portion define a locking element recess; wherein, when in a locked configuration, the at least one protrusion of the locking element is disposed in the locking element recess, thereby locking the first and second latch components in the latched configuration.

In yet another implementation, the present disclosure is directed to a closure assembly. The closure assembly includes a first latch component that includes a socket and a shelf defined by an inner surface of the socket; and a second latch component that includes a protruding extensible portion having first and second ends; wherein, when in a latched configuration, the extensible portion is inserted into the socket and the second end mechanically engages the shelf.

In yet another implementation, the present disclosure is directed to a pair of goggles. The goggles includes a goggle frame; a lens assembly that includes a lens frame and a lens coupled to an outer surface of the lens frame; and a closure assembly that includes a locking element, a first latch component coupled to the goggle frame and a second latch component coupled to the lens frame, the first and second latch components configured to mechanically engage to removably secure the lens assembly to the goggle frame; wherein one of the first and second latch components includes a socket and the other one of the first and second latch components includes a first portion; wherein when the first and second latch components are mechanically engaged, the first portion is inserted into and engages the socket and defines a locking element recess extending between the first and second latch components; wherein the locking element includes at least one protrusion configured to be inserted into the locking element recess to lock the first and second latch components together.

In yet another implementation, the present disclosure is directed to a pair of goggles. The goggles includes a goggle frame including a flexible frame ring that includes a plurality of ventilation openings; a foam layer coupled to an inner surface of the frame ring; and a fabric layer that is coupled to the frame ring and that encases the foam layer, wherein the fabric layer is designed to come into direct contact with a user's face, the fabric layer designed and configured to have a softness that improves user comfort and a durability that is greater than a durability of the foam layer to thereby increase a useful lifetime of the goggles.

In yet another implementation, the present disclosure is directed to a pair of goggles. The goggles includes a goggle frame; a lens assembly that includes a lens frame and a lens coupled to an outer surface of the lens frame; and a closure assembly that includes a locking element, a first latch component coupled to the goggle frame and a second latch component coupled to the lens frame, the first and second latch components configured to mechanically engage to removably secure the lens assembly to the goggle frame; wherein one of the first and second latch components includes a cross member having inner and outer surfaces and the other one of the first and second latch components includes a first surface and a laterally extending portion; wherein when the first and second latch components are mechanically engaged, the first surface engages the inner surface of the cross member, and the outer surface of the cross member and the laterally extending portion define a locking element recess; wherein the locking element includes a bottom surface and at least one protrusion disposed on the bottom surface, wherein the at least one protrusion is configured to be disposed in the locking element recess to lock the first and second latch components together in a locked configuration.

In yet another implementation, the present disclosure is directed to a pair of goggles. The goggles includes a goggle frame; a lens assembly that includes a lens frame and a lens coupled to an outer surface of the lens frame; and a closure assembly that includes a first latch component coupled to the goggle frame and a second latch component coupled to the lens frame, the first and second latch components configured to mechanically engage to removably secure the lens assembly to the goggle frame; wherein one of the first and second latch components includes a socket and the other one of the first and second latch components includes a first portion; wherein when the first and second latch components are mechanically engaged, the first portion is inserted into and engages the socket.

In yet another implementation, the present disclosure is directed to a method of using a lens change system of a pair of goggles. The method includes positioning a lens assembly on a goggle frame, the goggle frame including a first latch component and the lens assembly including a second latch component and a locking element; latching the first and second latch components together in a latched configuration; and locking the first and second latch components in the latched configuration, wherein the step of locking includes inserting a portion of the locking element into a recess defined by the first and second latch components, thereby preventing a relative movement between portions of the first and second latch components.

### BRIEF DESCRIPTION OF THE DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 is a front perspective view of a pair of goggles with a lens change system made in accordance with the present disclosure that includes at least one interchangeable lens assembly and a goggle frame;

FIG. 2 is a top perspective view of the goggles of FIG. 1 showing the lens assembly coupled to the goggle frame at a hinge connection at one end thereof during the installation or removal of the lens assembly;

FIG. 3 is a top view thereof;

FIG. 4 is an exploded view of the goggles of FIGS. 1-3;

FIG. 5A is a larger scale partial front view of the goggles of FIGS. 1-4 showing a lens closure assembly in a locked configuration;

FIG. 5B is a side view thereof;

FIG. 5C shows the closure assembly with a locking element pivoted to an unlocked position and showing first and second latch components of the closure assembly in a latched configuration;

FIG. 5D is a cross-sectional view of an alternate example of a closure assembly which is substantially the same as the closure assembly of FIGS. 1-5C except for the method of connection of the locking element to the second latch component;

FIG. 6A is a is a larger scale partial front view of the goggle frame of the goggles of FIGS. 1-4 with the lens assembly removed and showing the first latch component;

FIG. 6B is larger scale perspective view of the first latch component;

FIG. 6C is a larger scale partial front view of the first and second latch components in a latched configuration with the locking element removed;

FIGS. 7A-7D are a front perspective view, front view, side view, and rear perspective view, respectively, of the second latch component of the goggles of FIGS. 1-4;

FIGS. 8A-8C are a front view, rear view, and side view, respectively, of the locking element of the goggles of FIGS. 1-4;

FIG. 8D is a perspective view of the locking element coupled to the second latch component and pivoted to an unlocked position;

FIG. 9A is a front view of the goggle frame of the goggles of FIGS. 1-4;

FIG. 9B is a larger scale partial front view thereof;

FIG. 9C is a cross-sectional view of a portion of the goggles of FIGS. 1-4;

FIGS. 10A-10C are a rear perspective view, a bottom perspective view, and a side perspective view, respectively, of a hinge element of the lens assembly of the goggles of FIGS. 1-4;

FIGS. 11A and 11B are top and bottom views, respectively, of the goggles of FIGS. 1-4; and

FIG. 12 is a functional block diagram of an article that includes a closure assembly for securing together first and second parts of the article.

### DETAILED DESCRIPTION

Examples of closure assemblies for mechanically securing first and second portions of an article are described. In some examples, the closure assembly includes first and second latch components that mechanically engage and a locking element coupled to one of the latch components for locking the components in a latched configuration. The closure assemblies of the present disclosure can be made inexpensively, may be easier to recycle than many alternative arrangements, can be used quickly and easily even when wearing gloves and in extreme temperatures, and securely lock two components together. The closure assemblies of the present disclosure can be utilized on any article for securing two portions of the article together, such as enclosures (e.g. for securing a lid of a box, securing an opening or enclosure of a suitcase, duffle bag, backpack, drybag etc.), clothing items (e.g., such as a belt buckle, footwear buckle etc.), and sporting good items (e.g., eyewear lens change systems, helmet strap buckle, climbing harness, etc.). Closure devices as described herein can be used for connecting two parts, for example for connecting two belts, straps, cables, cords or the like or also as closure for a bag, a cover, a box, a glove box, or some other container. Closures as described herein can also be used for connecting other parts, for example for connecting objects on a backpack, a jacket or trousers, for fixing objects on a sports helmet, for fixing mobile phones or computers at holders, for fixing accessories such as e.g. a bicycle lock at a bicycle or for connecting other parts. Example implementations of closure assemblies incorporated in goggle lens change systems are described and illustrated herein, however, persons of ordinary skill in the art will appreciate the disclosed closure assemblies can be readily applied to any of a variety of other articles with little to no modification of the closure assembly.

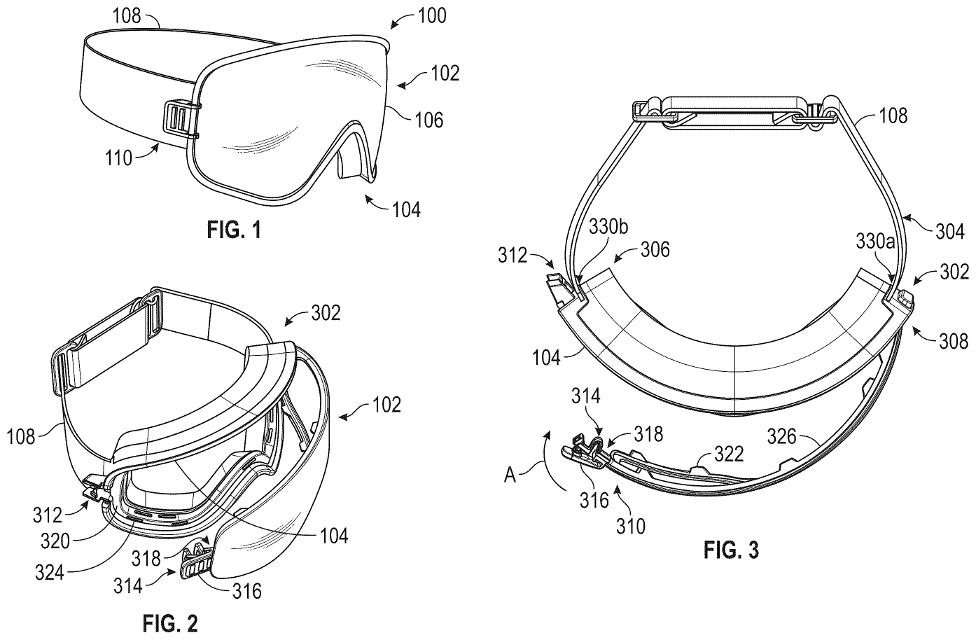

As shown in FIG. 1, an example goggle 100 includes a lens assembly 102 removably coupled to a goggle frame 104. Lens assembly 102 includes at least one lens, including an outer lens 106. Goggle 100 also includes a strap 108 for securing the goggles to a user's face and a closure assembly 110 for securing the lens assembly 102 to the goggle frame 104. FIGS. 2 and 3 show lens assembly 102 during an installation or removal process. As shown, in the illustrated example lens assembly 102 is designed and configured to be attached to goggle frame 104 by a first retention feature in the form of a hinge 302 located at a first side 304 of the goggle frame and a second retention feature in the form of closure assembly 110 located at an opposite second side 306 of the goggle frame. During installation, a hinge element of the lens assembly 102 may be coupled to a corresponding hinge element of the goggle frame 104 to form a hinge connection between a first end 308 of lens assembly 102 and first side 304 of goggle frame 104 and then the lens assembly 102 may be pivoted about the hinge connection in the direction of arrow A (FIG. 3) to couple a second end 310 of lens assembly 102 to second side 306 of the goggle frame.

Referring still to FIGS. 2 and 3, in the illustrated example, closure assembly 110 includes a first latch component 312 coupled to goggle frame 104 and a second latch component 314 coupled to lens assembly 102, wherein the first and second latch components are configured to mechanically engage to removably secure the lens assembly to the goggle frame. Closure assembly 110 also includes a locking element 316 in the form of a latch cover that is coupled to second latch component 314 at a pivot connection 318. In the illustrated example locking element 316 is configured to be pivoted to a locked position wherein it covers first and second latch components 312, 314 and locks them in a latched configuration.

Referring still to FIGS. 2 and 3, an outer surface of goggle frame 104 defines a lens seat 320 in the form of a recess that has a shape that is complementary to an outer shape of lens assembly 102 for seating the lens assembly in the goggle frame. In some embodiments, the lens assembly 102 and the goggle frame 104 may also include alignment features to facilitate alignment of the lens assembly 102 to the goggle frame 104. For example, as illustrated in FIGS. 2 and 3, the lens assembly 102 and the goggle frame 104 may include alignment elements in the form of extensions 322 (only one labeled) and corresponding recesses 324 (only one labeled) to facilitate alignment. In the illustrated example, recesses 324 are defined in a surface of lens seat 320 and spaced around a periphery of the lens seat. Extensions 322 protrude from an inner surface 326 of lens assembly 102 at locations that correspond to locations of recesses 324. Extensions 322 may be received in recesses 324 when lens assembly 102 is aligned with and being coupled to goggle frame 104 and facilitate proper alignment during the installation process. Once installed, the alignment features may also provide the additional benefit of restricting lateral movement of lens assembly 102 relative to goggle frame 104 thereby providing a more secure connection. In other embodiments, the recesses 324 may be defined in lens assembly 102, and the extensions 322 may be formed on the goggle frame 104, or there can be a combination of recesses and extensions disposed in both the lens assembly and goggle frame.

In the illustrated example, goggle 100 does not include any magnetic elements for alignment or coupling of lens assembly 102 and goggle frame 104. By omitting magnetic elements mixing of materials is minimized making the goggles easier to recycle. Omitting magnets along with other design features described herein also reduces the weight of the goggles, improving the user experience and reducing fatigue due to the lighter weight. The lighter weight also increases the ability of the goggles to stay in the correct position on the user's face during strenuous exercise, such as ski racing, skiing through a mogul field, etc. The omission of magnets also simplifies the manufacturing process and reduces manufacturing costs. It is a particular benefit of the structures disclosed herein that the lens assembly 102 can be coupled to the goggle frame 104 in a manner that is as fast and secure or more so than existing designs that require magnets. In alternative embodiments magnetic elements can be incorporated as is known in the art in combination with the closure assemblies disclosed herein and unless explicitly specified as excluding magnets the claims of the present application encompass both magnetic and magnet-free designs. In an example, discrete magnetic elements may be positioned around a periphery of the lens assembly 102 and goggle frame 104, such as adjacent to the alignment features (e.g. extensions 322 and recesses 324) or instead of the extensions and recesses for use in aligning and securing the lens assembly with the goggle frame as is known in the art.

Referring still to FIGS. 2 and 3, in the illustrated example the strap 108 is coupled to the goggles at locations that are inward or behind closure assembly 110 and hinge 302. The ends (not illustrated) of strap 108 are inserted into slots 330a, 330b in first side 304 and second side 306 of goggle frame 104 and anchored within the goggle frame. The illustrated method of attachment of strap 108 to goggle 100 provides a streamlined and low-profile appearance. In other examples strap 108 may be attached to goggle 100 in alternate ways known in the art, such as by outriggers coupled to the opposed sides 304, 306 of the goggles.

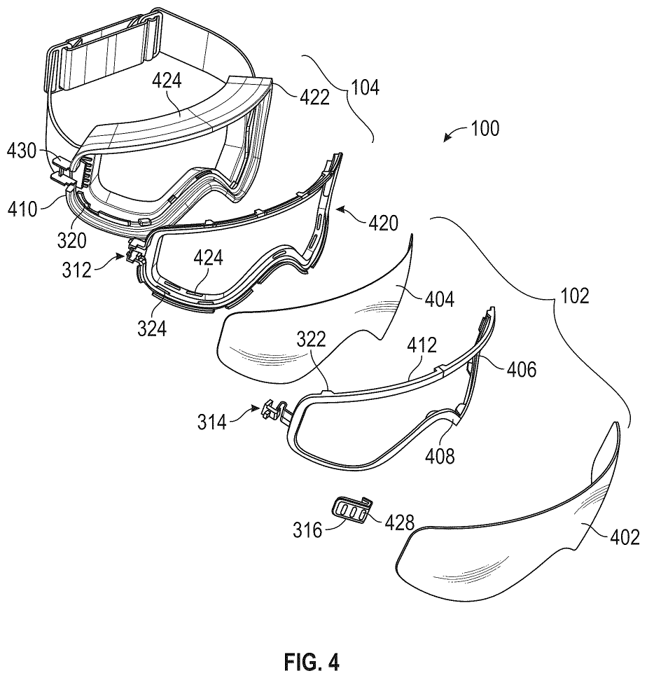

FIG. 4 is an exploded view of goggle 100 and shows the components of lens assembly 102, goggle frame 104, and closure assembly 110. In the illustrated example, lens assembly 102 is a dual lens structure including an outer lens 402 and inner lens 404 joined together by a lens frame 406. Outer lens 402 is attached to a front surface 408 of lens frame 406 and in the illustrated example has an outer dimension that is substantially the same or slightly larger than an outer dimension of the lens frame resulting in an outer edge of the lens frame being slightly recessed from an outer edge of the outer lens. Outer lens 402 has a shape and outer dimension that is complementary to a shape and outer dimension of lens seat 320 such that when assembled the outer lens is flush with an outer front edge surface 410 of goggle frame 104 and lens frame 406 is located behind the outer lens. Inner lens 404 is coupled to an inner surface 412 of lens frame 406 and in the illustrated example has an outer shape and dimension that is slightly smaller than an outer shape and dimension of the lens frame and is seated in an inner lens seat (not illustrated) formed in the inner surface of the lens frame 406 for a secure coupling of the inner lens to the lens frame. Extensions 322 (only one labeled) are located around an outer extent of the inner lens and extend inward of an inner surface of the inner lens for insertion into corresponding recesses 324 (only one labeled) in the goggle frame.

Second latch component 314 is coupled to one side of lens frame 406 and extends laterally therefrom. In the illustrated example, the second latch component 314 is directly coupled to an outer edge of the lens frame 406 and in some examples is integrally formed with the lens frame during an injection molding process. Both the second latch component 314 and the lens frame 406 are formed of a relatively hard or rigid material such as nylon, for example, TR90, and may have a durable, flexible, and lightweight construction.

Lenses 402, 404 may be of any material type and construction known in the art of eyewear lenses. For example, the lenses may be made from polycarbonate (PC), acrylic, or other materials. The lenses can have any shape known in the art, such as flat, curved, cylindrical, spherical, arcuate, compound etc. And the lenses can be configured according to any optical design to perform any optical function known in the art, such as tinting, filtering, polarization, laser protection, etc. and include any one or more corresponding optical coatings. The lenses may also be designed for any mechanical performance requirements known in the art such as scratch resistance, shatter resistance, projectile resistance, etc. By having an interchangeable lens assembly 102 a user can have a plurality of lens assemblies with different lens types for different applications or conditions, such as for low light and sunny conditions. As further described herein, closure assembly 110 is designed to make the exchange of lens assemblies 102 very easy such that the exchange can even be performed while a user is wearing the goggles and without removing his or her gloves. The lens assembly can, therefore, be changed on the fly and during use in response to changing conditions.

Referring still to FIG. 4, in the illustrated example goggle frame 104 includes an interface component 420 that is formed of a relatively hard or rigid material, such as nylon, and an assembly of other more flexible components that the interface component is coupled to and that provide a soft, flexible, and resilient interface between the user's head and the more rigid interface component 420 and lens assembly 102. In the illustrated example the more flexible components of the goggle frame 104 include a frame ring 422, one or more foam layers (not illustrated) coupled to an inner surface of the frame ring, and a fabric layer 424 that is coupled to the frame ring and encases the foam layers. In the illustrated example, an inner surface on a top side of frame ring 422 defines ventilation openings (not illustrated) which are covered by the fabric layer 424. On the opposite, bottom side of goggle frame 104, lower ventilation openings 426 (only one labeled) are defined by interface component 420 and an outer side of the lower ventilation openings are similarly covered by the fabric layer.

Fabric layer 424 is designed to come into direct contact with a user's face, may have a softness that improves user comfort and may also be more durable than the foam layer, which has the advantage of increasing the usable lifetime of the goggles. For example, without fabric layer 424, the foam layers may begin to deteriorate, which commonly occurs with prior art goggles and can often be a primary reason why a user chooses to dispose of the goggles. By encasing the foam layer(s) in fabric 424, deterioration of the foam is prevented. The frame ring 422, foam layers, and fabric layer can be formed from a variety of different types of materials, for example, the frame ring may be formed from thermoplastic polyurethane (TPU), the foam layers may be formed from polyurethane and the fabric layer may be formed from polyester, for example, a fleece material, for example, greater than 90% or greater than 95% or 100% polyester.

As shown in FIG. 4, first latch component 312 is coupled to one side of interface component 420 and extends laterally therefrom. In the illustrated example, the first latch component 312 is directly coupled to an outer edge of the interface component 420 and in some examples is integrally formed with the interface component during an injection molding process. Both the first latch component 312 and the interface component 420 are formed of a relatively hard or rigid material such as nylon, for example, TR90, and may have a rigid, durable, flexible, and lightweight construction.

In the illustrated example, first and second latch components 312, 314 are, therefore, both formed from relatively rigid materials which provides a rigidness to the locking system of the closure assembly 110. As further described below, the first and second latch components 312, 314 are designed and configured to mechanically engage, e.g., with corresponding engagement features, when in a latched configuration.

In addition to first and second latch components 312, 314, closure assembly 110 also includes locking element 316. In the illustrated example, locking element 316 is formed from a relatively soft and flexible and resilient polymer material, such as a type of TPU, such as M90. In an example, locking element 316 is formed from a material that remains relatively flexible and ductile at low temperatures, such as temperatures as low as −20° C. Locking element 316 may provide one or more of a plurality of functions. For example, locking element 316 locks the first and second latch components 312, 314 in a latched configuration. Locking element 316 may also be in the form of a latch cover and substantially cover the first and second latches when lens assembly 102 is installed on goggle frame 104. Locking element 316 may be an oversized flexible tab and also function as a pull tab for easily grasping and removing lens assembly 102 which can help avoid the need to touch lenses 402, 404 during installation and removal. In the illustrated example, locking element 316 is relatively oversized to facilitate operation of the closure assembly 110 even when wearing gloves and in challenging conditions, such as below freezing temperatures. Locking element 316 also includes finger-engaging contours 428 located on a top surface of the locking element to improve the ability to grasp the locking element during installation and removal of the lens assembly 102.

Frame ring 422 may also include a closure assembly housing 430 that may be flexible and formed from the same material as the frame ring, such as TPU. In the illustrated example, first latch component 312 is located in a recess defined by the closure assembly housing 430. An outer shape and dimension of the closure assembly housing is substantially the same as an outer dimension of locking element 316 such that when in a locked configuration the closure assembly housing 430 and locking element 316 substantially enclose the first and second latch components 312, 314 and provide a resilient outer layer to the closure assembly 110 that is free of any protrusions that could be caught or snagged during use of the goggles.

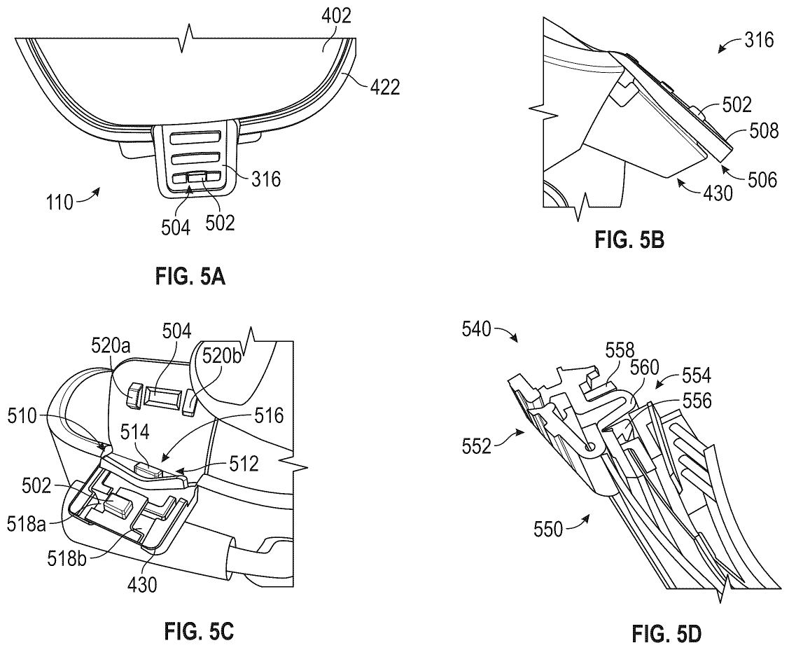

FIGS. 5A-5D show larger scale views of closure assembly 110, wherein FIG. 5A is a front view of the closure assembly in a locked configuration, FIG. 5B is a side view thereof, FIG. 5C is a front-side-perspective view of the closure assembly in an unlocked latched configuration and FIG. 5D is a top cross sectional view of an alternate example of a closure assembly. As shown in FIG. 5A, when in the locked configuration locking element 316 is coupled to second latch component 314 with a first upwardly projecting element 502 pressed through a first opening 504 in the locking element for securing the locking element in the locked configuration. A distal end of element 502 may have a width that is greater than a width of opening 504, such that the upwardly projecting element is resiliently pressed through the opening when engaging the two components. As shown in FIG. 5B, when in the locked configuration a bottom surface 506 of the locking element is flush with a top mating surface of the closure assembly housing 430 substantially encasing the latch components 312, 314 therein. Locking element 316 may have a length that is greater than an outer dimension of the housing 430 in a lateral direction resulting in a first end 508 of locking element 316 protruding beyond the housing to facilitate pressing the locking element upward, even when wearing gloves, for disengaging the locking element from the first and second latch components.

As shown in FIG. 5C, a second end 510 of locking element 316 may be coupled to second latch component 314 by a pivot connection. In the illustrated example, the pivot connection includes a living hinge 512 formed in a portion of the locking element proximate second end 510. In the example, shown in FIG. 5C, the locking element 316 is coupled to the second latch component 314 by a second upwardly projecting element 514 pressed through a second opening 516 in the locking element.

Referring still to FIG. 5C, the first and second latch components 312, 314 in the latched configuration may form first and second locking element recesses 518a, 518b. In the illustrated example, locking element 316 includes two protrusions 520a, 520b that are configured and dimensioned to be disposed in corresponding ones of the locking element recesses. As discussed more below, by inserting protrusions 520 into recesses 518, locking element 316 locks the first and second latch components in the latched configuration thereby preventing the unlatching or decoupling of the closure assembly and securely coupling lens assembly 102 to goggle frame 104.

FIG. 5D shows an alternate embodiment of a closure assembly 540 that is substantially the same as closure assembly 110 shown in FIGS. 1-5C except that second end 550 of locking element 552 is coupled to second latch component 554 by a protrusion 556 extending from a bottom surface of the locking element that is inserted into a corresponding opening in the second latch component, rather than the opposite (upwardly projecting element 514 of latch component extending through opening 516 in locking element) shown in, for example, FIG. 5C. FIG. 5D also illustrates a nested arrangement of second latch component 554 with a first latch component 558 where a downwardly protruding portion 560 of second latch component 554 is disposed in a socket defined by first latch component 558.

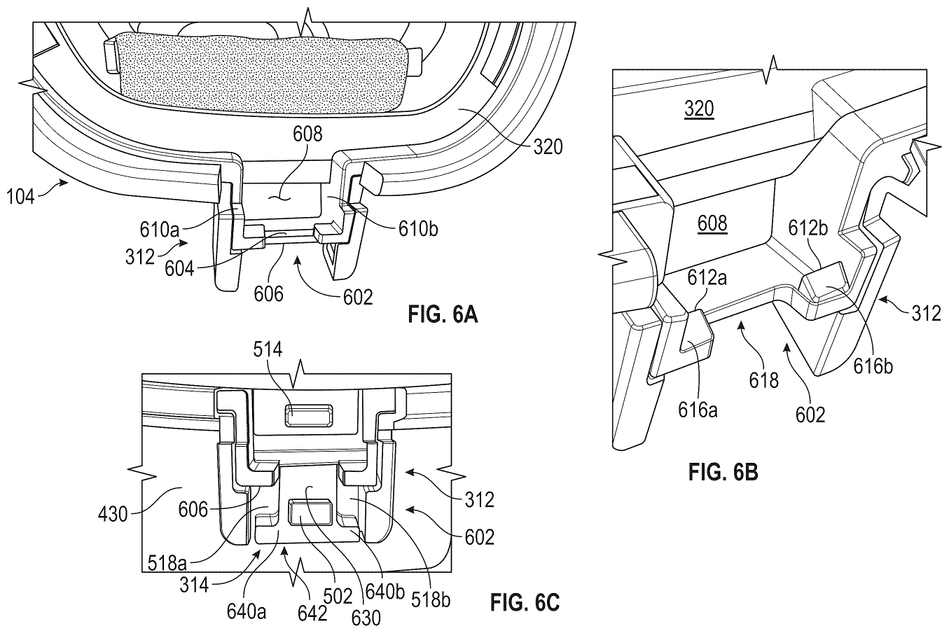

FIG. 6A is a front perspective view of a portion of goggle 100 with lens assembly 102 removed, showing a portion of goggle frame 104, including lens seat 320, and first latch component 312. In the illustrated example, first latch component 312 includes a cross member 602 having an inner surface 604 and an opposed outer surface 606. Cross member 602 partially defines a socket 608. First latch component 312 may also include opposed laterally extending members 610a and 610b that together with cross member 602 define socket 608. Cross member 602 may be laterally offset from an outer extent of lens seat 320.

FIG. 6B is a larger scale perspective view of first latch component 312. Inner surface 604 of cross member 602 may define at least one shelf and in the illustrated example defines a pair of shelves 612a, 612b. In the illustrated example, shelves 612 are defined by two corresponding inwardly extending protrusions. In the illustrated example, the inwardly extending protrusions have beveled top surfaces 616a, 616b to facilitate sliding engagement with portions of second latch component 314 as discussed more below. First latch component may also include a tab recess 618 that is configured and dimensioned to receive a tab portion 630 (e.g., FIG. 6C) of second locking element 314.

FIG. 6C is a larger scale front view of closure assembly 110 with lens assembly 102 installed and first and second latch components 312, 314 in a latched configuration, and with locking element 316 removed. In the latched configuration a portion of second locking element 314 is inserted into and mechanically engaged with socket 608. The mechanical engagement may include engagement between a portion of second latch component and the at least one shelf 612 of first latch component 312 (see, e.g., FIG. 6B). Tab 630 of second latch component 314 is disposed in tab recess 618 of first latch component 312. Second latch component 314 may include a laterally extending portion 640 coupled to tab portion 630, in the illustrated example the laterally extending portion includes a pair of laterally extending portions 640a, 640b that extend laterally from both sides of the tab portion. An end 642 of second latch component 314 is configured and dimensioned as a finger engaging portion and to be depressed, for example, in an inward and upward direction for disengaging the first and second latch components 312, 314 from the latched configuration for removal of the lens assembly 102 from the goggle frame 104.

Laterally extending portions 640 and outer surface 606 of cross member 602 at least partially define locking element recesses 518a, 518b. As discussed above in connection with FIG. 5C, when in the locked configuration, protrusions 520 of locking element 316 (see FIG. 5C) may be disposed in locking element recesses 518, thereby locking the first and second latch components 312, 314 in the latched configuration. More specifically, with protrusions 520 so disposed, second latch component 314 is prevented from being compressed or bent in an inward direction, thereby preventing the disengagement of the second latch component from first latch component 312. As noted above, protrusions 520 may be formed from a relatively soft and flexible polymer material and they may have a size that is slightly larger than a size of locking element recesses 518. As also noted above first and second latch components 312, 214 may be formed from a more rigid material, resulting in the locking element recesses being rigid. Insertion of the resilient protrusions 520 into the rigid recesses 518 results in a resilient compression of the protrusions. Also, the flexible and resilient nature of the protrusions provides a degree of compliance between the first and second latch components when they are in the latched and locked configuration.

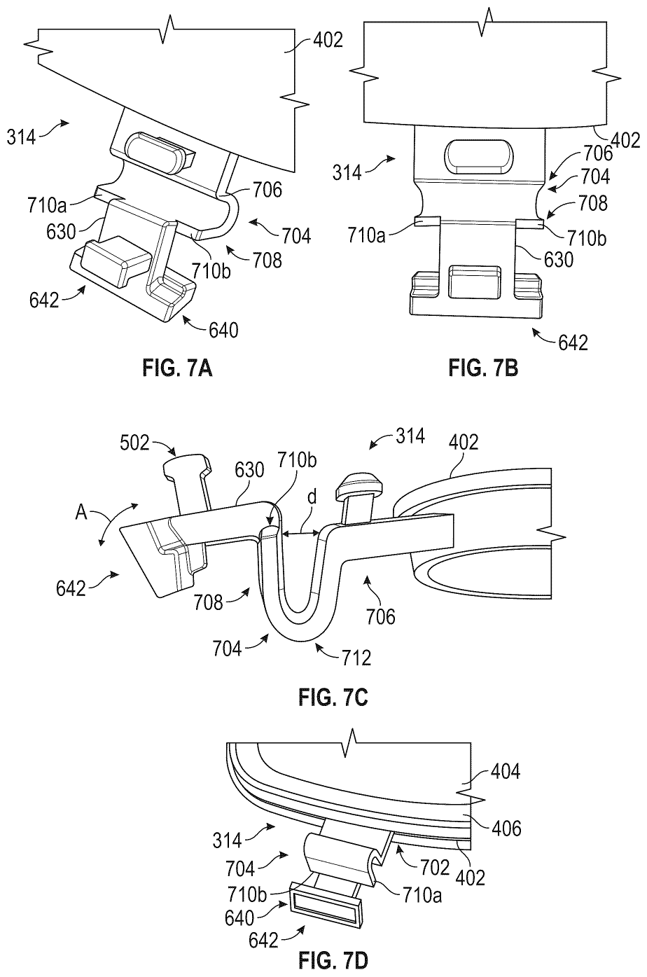

FIGS. 7A-7D illustrate one example implementation of second latch component 314. In the illustrated example, second latch component 314 extends laterally from a side of lens frame 406 with a first end 702 of the second latch component coupled to the lens frame. Second latch component 314 includes an extensible portion 704 that includes a first end 706 and a second end 708, the second end defining a first surface 710. In the illustrated example, first surface 710 includes two portions 710a, 710b located on opposite sides of tab 630. Second end 642 may be designed and configured as a finger-engaging portion and in the illustrated example includes a recess or other contour to facilitate engagement of a user's hand with the second latch component.

With continuing reference to FIGS. 7A-7D as well as FIGS. 6B and 6C, extensible portion 704 is configured to be inserted into socket 608 for mechanical engagement therebetween.

In the illustrated example, the mechanical engagement includes engagement of first surface 710 with inner surface 604 of cross member 602, and more specifically, engagement of the first surface 710 with shelf 612. A method of installation of lens assembly 102 may include inserting extensible portion 704 into socket 608 and pressing, extending, or bending the first latch component to engage the first latch component, e.g., second end 708, with the second latch component, e.g., shelves 612. In an example, extensible portion 704 is designed and configured to enable second end 642 to pivot, extend, and/or flex relative to first end 702 and lens frame 406 to facilitate the mechanical coupling. During the installation first upwardly projecting element 502 may serve as a finger engaging portion or knob for both pressing and pulling the second latch component, e.g., end 708, into engagement with the first latch component.

In the illustrated example, extensible portion 704 is a living hinge and forms a downwardly projecting portion of the second latch component 314. Extensible portion 704 facilitates a bending of second latch component 314 in the direction indicated by arrow A in FIG. 7C generally about an apex 712 of a curved, bent, or u-shaped portion of the second latch component that forms the extensible portion. The bending (in a downward direction when viewed from the side as in FIG. 7C) results in a distance, d, between the first and second ends 706, 708 increasing and the pulling of the second end 708 into engagement with the first latch component. As shown, the second latch component may extend laterally from the lens frame in a first plane, wherein the extensible portion is a bent portion of the second latch component having an apex 712 and protruding in a direction substantially perpendicular to the first plane.

A method of decoupling or unlatching first and second latch components 312, 314 may include pressing second end 642 in a direction that is inward and/or towards a front side of goggle 100 (in a generally upward and inward direction when viewed from the side as in FIG. 7C). The pressing of second end 642 may include a bending or compression of second latch component 314, for example, at extensible portion 704, resulting in a compression of the extensible portion and a decrease in the distance, d, between first and second ends 706, 708. The compression of the extensible portion and laterally inward movement of second end 708 results in the decoupling or unlatching of the first and second latch components.

Locking element 316 is designed to lock the first and second latch components 312, 314 in the latched configuration by preventing the compression of the extensible portion 704. More specifically, in the illustrated example, when protrusions 520 (FIG. 5C) of locking element 316 are disposed in locking element recesses 518 (FIG. 6C), the protrusions are in abutting contact with laterally projecting portion 640 of the second locking element and cross member 602 of the second locking element. So positioned the protrusions prevent the inward bending of the second latch component 314 and the inward movement of second end 708, thereby preventing the decoupling of the first and second latch components.

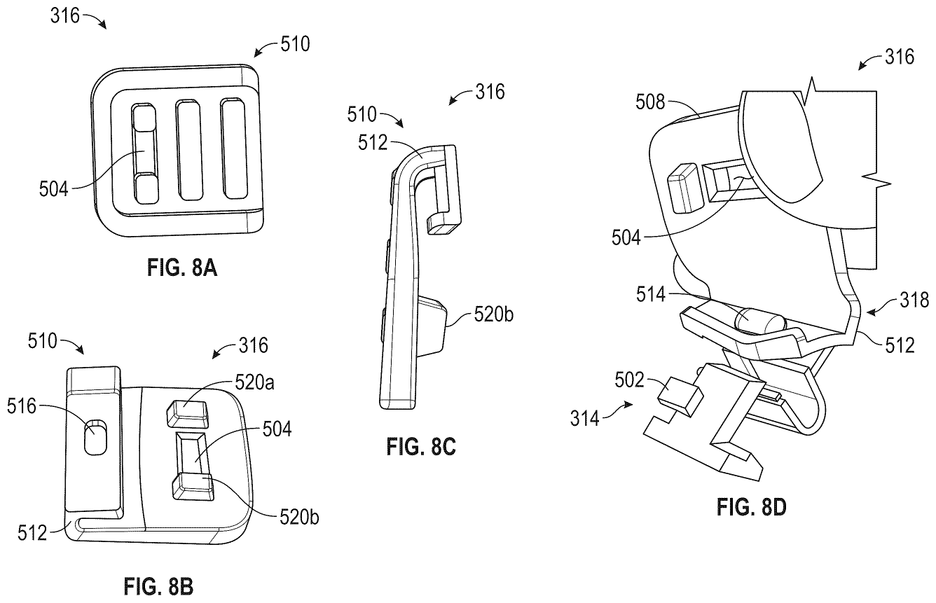

FIGS. 8A-8C show one example implementation of locking element 316 and FIG. 8D shows the locking element coupled to second latch component 314. In the illustrated example, locking element 316 has an oversized and generally square shape and is designed as a cover that in cooperation with latch housing 430 encases the first and second latch components. Locking element 316 includes living hinge 512 at second end 510 and as shown in FIG. 8D may be coupled to second latch component 314 by pressing upwardly projecting element 514 through opening 516. In the illustrated example, protrusions 520 are aligned with and in close proximity to opening 516 which increases the structural integrity of the locking mechanism. More specifically, when in a locked configuration locking element 316 is secured to first latch component 314 by the engagement of upwardly projecting element 502 with opening 504. Due to the relatively flexible and pliable nature of locking element 316 it can be beneficial to locate protrusions 520 closely adjacent the anchor point (opening 504) to ensure the fixation of the protrusions within locking element recesses 518 (FIG. 5C).

FIG. 8D shows locking element 316 pivoted about pivot connection 318 to an open position (FIG. 8D showing a user's thumb bending the tab open). In the example pivot connection 318 is formed by living hinge 512. As noted above in connection with FIG. 5B, when in the locked configuration first end 508 protrudes slightly beyond housing 430 allowing a user to easily engage and press the locking element up to the position shown in FIG. 8D. Locking element 316 then provides an additional function as an oversized pull tab as shown in FIG. 8D, allowing a user to easily grasp the lens assembly 102 without touching the lenses 402, 404. In FIG. 8D a curvature is visible along a length of the locking element 316 illustrating the resilient and flexible nature of the construction of the locking element in the illustrated example. As noted above, in some examples the locking element may be formed from a material that is designed and selected to retain flexibility and resilience over a wide range of temperatures to facilitate the functionality of the closure assembly in relatively cold and harsh conditions, for example to temperatures down to at least −20° C.

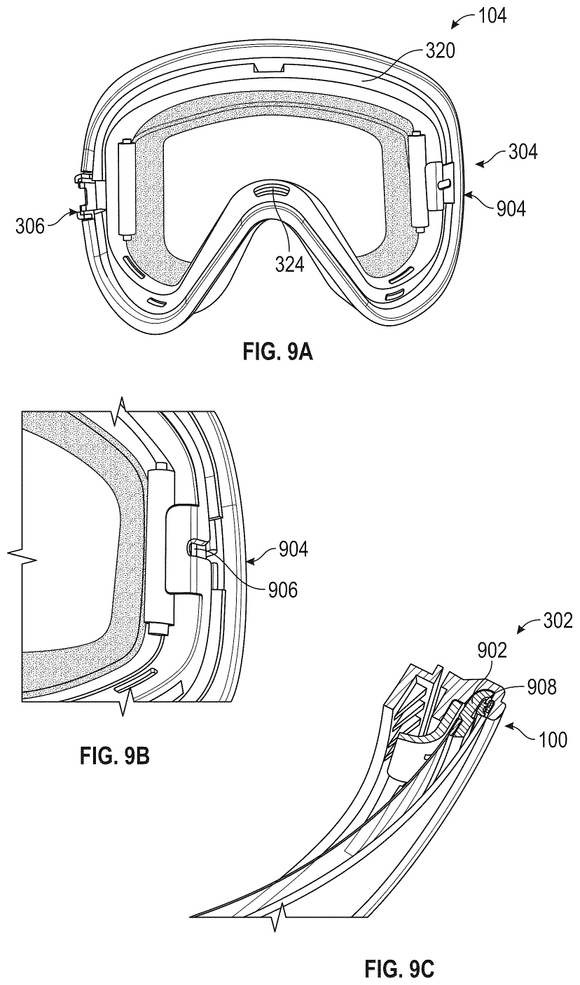

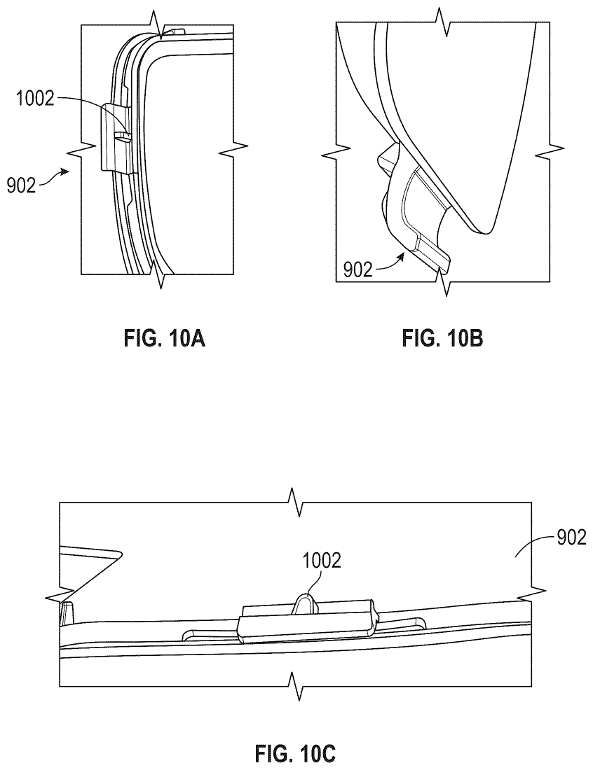

Turning to FIGS. 9A-10C, in the illustrated example lens assembly 102 may be attached to goggle frame 104 by a first retention feature in the form of a hinge 302 located at a first side 304 of the goggle frame and a second retention feature in the form of closure assembly 110 located at an opposite second side 306 of the goggle frame. During installation, a first hinge element 902 of the lens assembly 102 is coupled to a corresponding second hinge element 904 of the goggle frame 104 to form a hinge connection between a first end 308 of lens assembly 102 and first side 304 of goggle frame 104 and then the lens assembly 102 is pivoted about the hinge connection to couple a second end 310 of lens assembly 102 to second side 306 of the goggle frame. FIGS. 9A-10C illustrate one example implementation of hinge 302. As shown in FIGS. 9A and 9B, second hinge element 904 may be a recess formed in first side 304 of goggle frame 104 and in the illustrated example is formed in interface component 420 of the goggle frame. Second hinge element 904 may also include a laterally extending channel 906 for guiding first hinge element 902 into hinge recess 904 and guiding the lens assembly in the proper orientation during the lens installation process. As shown in FIGS. 9C and 10A-10C, first hinge element 902 may be a laterally extending member with an upwardly curved end extending from a side of lens assembly 102. First hinge element 902 may also include a downwardly projecting elongate rib 1002 for engaging complementary-shaped channel 906 in hinge recess 904. As best seen in FIG. 9C, the curved contour of first hinge element 902 may be designed and configured to be positioned against a complementary-shaped curved member 908 of second hinge element 904 that forms a lock that prevents movement of the lens assembly 102 in a lateral direction away from first side 304 of goggle frame 104. First and second hinge elements 902, 904, therefore, form a hinge lock that secures first end 308 of lens assembly 102 to first side 304 of goggle frame 104.

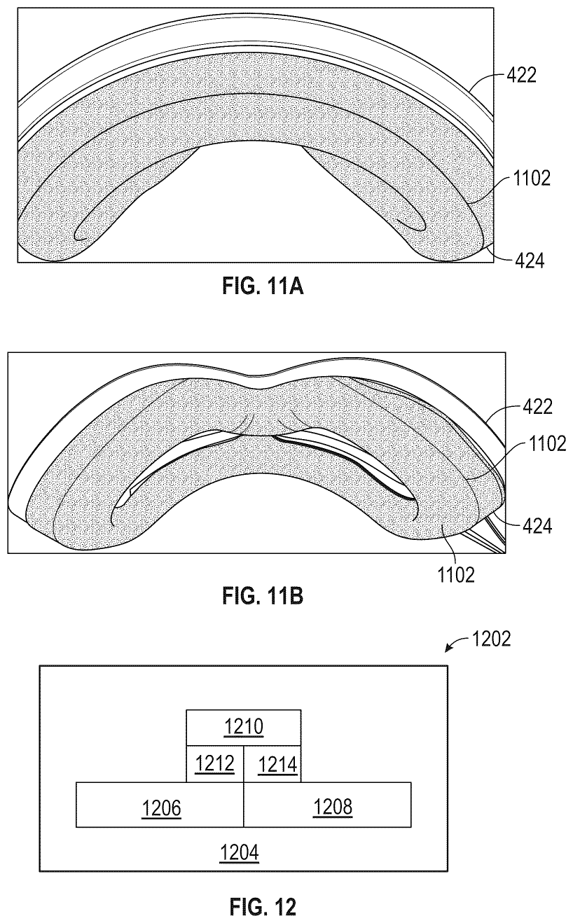

FIGS. 11A and 11B are top and bottom views of goggle 100 and show one example of fabric layer 424. As shown and described above, fabric layer 424 encases foam layers (not visible) located on an inner side of frame ring 422. In the illustrated example, fabric layer 424 includes an indented line 1102 that extends around a circumference of the goggles and generally demarcates the interface between frame ring 422 and the foam layers. As noted above, fabric layer 424 provides both aesthetic and functional benefits by providing a sleek simplified and streamlined appearance while also preventing the deterioration of the foam layers, thereby extending the useful life of the goggles.

FIG. 12 is a functional block diagram of another example of a closure assembly 1202 made in accordance with the present disclosure and conceptually shows the closure assembly 1202 disposed on an article 1204 and used to secure a first part 1206 of the article to a second part 1208 of the article. Closure assembly 1202 may have the same or similar construction as closure assembly 110, including a locking element 1210, a first latch component 1212 and a second latch component 1214. Article 1204 may be virtually any article having first and second portions capable of securement by closure assembly 1202. Non-limiting examples include but are not limited to an enclosure for securing a lid of a box, securing an opening or enclosure of a suitcase, duffle bag, backpack, drybag etc., a clothing item (e.g., such as a belt buckle, footwear buckle etc.), or a sporting good item (e.g., eyewear lens change systems as disclosed herein wherein first and second parts 1206, 1208 are lens assembly 102 and goggle frame 104 of goggle 100), a helmet strap buckle, climbing harness, etc.). First and second parts 1206, 1208 may be two belts, straps, cables, cords or the like or portions of a closure for a bag, a cover, a box, a glove box, or some other container. Closures as described here can also be used for connecting other parts, for example for connecting objects at article 1204, wherein the article may be a backpack, a jacket or trousers, or sports helmet, for fixing mobile phones or computers at holders, for fixing accessories such as e.g. a bicycle lock at a bicycle or for connecting other parts.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure.

Accordingly, this description is meant to be taken only by way of example, and not to otherwise limit the scope of this disclosure.

What is claimed is:

1. A pair of goggles, comprising:
a goggle frame;
a lens assembly that includes a lens frame and a lens coupled to an outer surface of the lens frame; and
a closure assembly that includes a locking element, a first latch component coupled to the goggle frame and a second latch component coupled to the lens frame, the first and second latch components configured to mechanically engage to removably secure the lens assembly to the goggle frame; wherein one of the first and second latch components includes a socket and the other one of the first and second latch components includes a first portion;
wherein when the first and second latch components are mechanically engaged, the first portion is inserted into and engages the socket and defines a locking element recess extending between the first and second latch components;
wherein the locking element includes at least one protrusion configured to be inserted into the locking element recess to lock the first and second latch components together; and
wherein the second latch component includes an extensible portion for resiliently expanding or bending the first portion for engagement with the socket.

2. The goggles of claim 1, wherein the extensible portion includes first and second ends, wherein the second end of the extensible portion mechanically engages an inner surface of the socket.

3. The goggles of claim 1, wherein the extensible portion is a downwardly projecting living hinge.

4. The goggles of claim 2, wherein the inner surface of the socket includes a shelf, wherein the second end of the extensible portion couples to the shelf.

5. The goggles of claim 4, wherein an end of the second latch component includes a finger engaging portion, wherein the first and second latch components are decoupled by pressing the finger engaging portion, thereby compressing the extensible portion and decoupling the first portion from the socket.

6. The goggles of claim 1, wherein the goggle frame includes a plurality of first lens alignment elements spaced around a periphery of the goggle frame and the lens assembly includes a plurality of second lens alignment elements spaced around a periphery of the lens frame, wherein the first and second alignment elements include at least one of complementary extensions and recesses and magnetic elements.

7. A pair of goggles, comprising:
a goggle frame;
a lens assembly that includes a lens frame and a lens coupled to an outer surface of the lens frame; and
a closure assembly that includes a locking element, a first latch component coupled to the goggle frame and a second latch component coupled to the lens frame, the first and second latch components configured to mechanically engage to removably secure the lens assembly to the goggle frame; wherein one of the first and second latch components includes a cross member having inner and outer surfaces and the other one of the first and second latch components includes a first surface and a laterally extending portion;
wherein when the first and second latch components are mechanically engaged, the first surface engages the inner surface of the cross member, and the outer surface of the cross member and the laterally extending portion define a locking element recess;
wherein the locking element includes a bottom surface and at least one protrusion disposed on the bottom surface, wherein the at least one protrusion is configured to be disposed in the locking element recess to lock the first and second latch components together in a locked configuration.

8. A pair of goggles, comprising:
a goggle frame;
a lens assembly that includes a lens frame and a lens coupled to an outer surface of the lens frame; and
a closure assembly that includes a first latch component coupled to the goggle frame and a second latch component coupled to the lens frame, the first and second latch components configured to mechanically engage to removably secure the lens assembly to the goggle frame;
wherein one of the first and second latch components includes a socket and the other one of the first and second latch components includes a first portion;
wherein when the first and second latch components are mechanically engaged, the first portion is inserted into and engages the socket, wherein the first portion is a protruding extensible portion having first and second ends, wherein the second end engages an inner surface of the socket, wherein the second latch component extends laterally from the lens frame in a first plane, wherein the extensible portion is a bent portion of the second latch component having an apex and protruding in a direction substantially perpendicular to the first plane.

9. A method of using a lens change system of a pair of goggles, the method comprising:
positioning a lens assembly on a pair of goggles frame, the goggle frame including a first latch component and the lens assembly including a second latch component and a locking element;
latching the first and second latch components together in a latched configuration; and
locking the first and second latch components in the latched configuration, wherein the step of locking includes inserting a portion of the locking element into a recess defined by the first and second latch components, thereby preventing a relative movement between portions of the first and second latch components, wherein the step of latching the first and second latch components together includes inserting a first portion of the second latch component into a socket of the first latch component, wherein the first portion includes first and second ends, wherein the step of latching further includes bending the second latch component at the first portion, thereby separating the first and second ends and mechanically engaging the second end with a portion of the socket.

10. The method of claim 9, further comprising removing the lens assembly by compressing the first portion to thereby decouple the second end from the portion of the socket.

11. The method of claim 9, wherein the lens assembly and goggle frame each have first and second opposite ends, the first and second latch components located at the first ends of the goggle frame and lens assembly, wherein the step of positioning the lens assembly on the goggle frame includes: forming a hinge connection between the second ends of the lens assembly and goggle frame; and pivoting the lens assembly about the hinge connection.

12. A closure assembly comprising:
a first latch component that includes a socket;
a second latch component that includes a first portion;
a locking element coupled to the second latch component, a bottom surface of the locking element having at least one protrusion;
wherein, when in a latched configuration, the first portion is inserted into and mechanically engages the socket;
wherein, when in a locked configuration, the at least one protrusion of the locking element is disposed in a locking element recess extending between the first and second latch components to thereby lock the first and second latch components in the latched configuration, wherein the first portion includes an extensible portion for resiliently expanding or bending the first portion for engagement with the socket.

13. A closure assembly comprising:
a first latch component that includes a cross member having inner and outer surfaces;
a second latch component that includes a first surface and a laterally extending portion;
a locking element coupled to the second latch component by a pivot connection, a bottom surface of the locking element having at least one protrusion;
wherein, when in a latched configuration, the first surface engages the inner surface of the cross member, and the outer surface of the cross member and the laterally extending portion define a locking element recess;
wherein, when in a locked configuration, the at least one protrusion of the locking element is disposed in the locking element recess, thereby locking the first and second latch components in the latched configuration.

14. A closure assembly comprising:
a first latch component that includes a socket and a shelf defined by an inner surface of the socket; and
a second latch component that includes a protruding extensible portion having first and second ends;
wherein, when in a latched configuration, the extensible portion is inserted into the socket and the second end mechanically engages the shelf, wherein the extensible portion is a living hinge, wherein the closure assembly further comprises a locking element coupled to the second latch component, a bottom surface of the locking element having at least one protrusion;
wherein, when the first and second latch components are in the latched configuration the first and second latch components define a locking element recess extending therebetween respective portions thereof;
wherein when in a locked configuration, the at least one protrusion of the locking element is disposed in a locking element recess extending between the first and second latch components to thereby lock the first and second latch components in the latched configuration

15. A sporting good item, comprising:
first and second portions; and
the closure assembly of claim 12, wherein the first and second latch components and locking element are coupled to corresponding ones of the first and second portions for coupling the first and second portions, wherein the sporting good item is:
goggles and the first and second portions are a pair of goggles frame and a lens assembly for reliably coupling the lens assembly to the goggle frame;
a helmet and the first and second portions are first and second portions of a chin strap or a body of the helmet and an attachable accessory for the helmet; or
a pack, including a duffle bag or backpack.
